(19) United States
(12) Patent Application Publication
(10) Pub. No.: US 2009/0221985 A1
(43) Pub. Date: Sep. 3, 2009

(54) SYSTEM FOR IONTOPHORETIC TRANSDERMAL DELIVERY OF POLYMERIC AGENTS AND METHODS OF USE THEREOF

(76) Inventors: Shmuel Bukshpan, Ramat Hasharon (IL); Gleb Zilberstein, Rehovot (IL)

Correspondence Address: WINSTON & STRAWN LLP;PATENT DEPARTMENT, 1700 K STREET, N.W., WASHINGTON, DC, 20006, US

(21) Appl. No.: 12/090,675

(22) PCT Filed: Oct. 19, 2006
(86) PCT No.: PCT/IL06/01206

§ 371 Date: Nov. 5, 2008

Related U.S. Application Data
(60) Provisional application No. 60/727,828, filed on Oct. 19, 2005.

(51) Int. Cl. A61N 1/30; A61K 38/02; A61K 31/728; A61K 38/39

(52) U.S. Cl. 604/501; 514/2; 514/54; 514/12

(57) ABSTRACT

A system for iontophoretic transdermal delivery of a non-peptidic polymeric cosmetic or therapeutic agent or of a polypeptide useful for delivery of high molecular weight cosmetic or therapeutic agents. Particularly, the present invention relates to a system that includes an iontophoresis device and a pharmaceutical composition of a non-peptidic polymeric cosmetic or therapeutic agent together with a peptide, polypeptide or protein and a pharmaceutically acceptable carrier having a pH below or above the isoelectric pH of the peptide, polypeptide or protein.

### FIELD OF THE INVENTION

The present invention relates to a system for iontophoretic transdermal delivery of a non-peptidic polymeric agent together with a peptide, polypeptide or protein, the system being particularly useful for transdermal delivery of high molecular weight cosmetic or therapeutic agents.

### BACKGROUND OF THE INVENTION

The therapeutic and cosmetic utility of topically applied chemical agents is limited by the lipid-rich stratum corneum, the thin outermost layer of the skin that acts as a highly resistant lipid barrier to penetration of chemical agents into the skin. In both the pharmaceutical and cosmetic arenas, significant efforts have been invested in attempts to overcome the skin's natural barrier to topical delivery of functional agents into the skin or into systemic circulation.

Generally, three primary routes across the stratum corneum are available for molecular transport: (1) Normal or chemically modified skin allows diffusion of small molecules, usually following a tortuous intercellular path within the lipids of the stratum corneum; (2) Transcellular pathways crossing both the cells and intercellular lipids of the stratum corneum can be created by electroporation to allow passage of chemical compounds; and (3) “Shunt” pathways through the hair follicles and sweat ducts may be utilized during iontophoresis (IPH), pressure-mediated delivery, and liposomal transport. It is also known that while electroporation involves the use of invasive electrodes and strong electrical pulses, iontophoresis is relatively non-invasive, well tolerated, and is developed for use in conscious or ambulatory patients.

The main factors causing skin aging are natural processes (such as aging), lifestyle factors (such as smoking), and environmental stressors (such as UV radiation, chemical pollutants, etc.). It is now medically recognized that many of these factors damage skin through production of oxy-radical damage. Superoxide and the subsequently generated hydrogen peroxide and hydroxyl radical have been shown to effect skin and other tissues by destroying lipid membranes, breaking down DNA, inactivating enzymes, and the like. As a result of this damage, certain anatomical changes occur, including thinning of the epidermis, thickening of the stratum corneum, reduction of blood supply to the skin, loss of collagen, and formation of age spots, lines and wrinkles.

To date no satisfactory methods are available to reverse damage to the skin due to aging and exposure to environmental factors and related toxic insults.

U.S. Patent Application Publication No. 2004/0225276 discloses methods of administering a material to a patient for dermal enhancement and/or as soft tissue fillers. The method of administering according to U.S. Patent Application Publication No. 2004/0225276 comprises steps of injecting the material into the patient. Among the materials suitable for dermal enhancement are hyaluronic acid, hydrogel, polylactic acid and collagen.

U.S. Patent Application Publication No. 2005/0191252 discloses a skin beautification system comprising an ultrasonic vibrator, an iontophoresis device, and a cosmetic additive. According to U.S. Patent Application Publication No. 2005/0191252, the ultrasonic vibrator may contain a high ionization iontophoresis device. The skin beautification system according to U.S. Patent Application Publication No. 2005/0191252 provides an effective action of the cosmetic additive. The cosmetic additive may be a moisturizing cosmetic additive such as hyaluronic acid, collagen, placental extract or lactic acid.

U.S. Pat. No. 6,432,710 discloses a composition for repairing tissue that has degenerated in a subject as a result of a disease, disorder, or a defect, the composition comprises a biodegradable acellular matrix and autologous fibroblasts. U.S. Pat. No. 6,432,710 further discloses methods of repairing tissue comprising a step of placing a composition comprising a biodegradable acellular matrix and autologous fibroblasts on a site of tissue degeneration. The biodegradable acellular matrix can comprise collagen, gelatin, polyglycolic acid or hydroxyapatite.

U.S. Pat. No. 5,961,482 discloses an iontophoretic device for transdermal systemic administration of ionized pharmaceutical compositions, preferably peptide compositions. The device according to U.S. Pat. No. 5,961,482 is preferably a wrist-band type.

The iontophoretic delivery methods known in the art have limited capability for delivering polymeric agents transdermally. Thus, there is an urgent need for improved iontophoretic delivery methods capable of delivering polymeric agents transdermally.

### SUMMARY OF THE INVENTION

The present invention provides a system for iontophoretic transdermal delivery of cosmetic or pharmaceutical compositions comprising as active ingredients a non-peptidic polymeric agent together with a peptide, polypeptide or protein and a pharmaceutically acceptable carrier, wherein the pharmaceutically acceptable carrier endows a positive or negative charge to the peptide, polypeptide or protein. The present invention further provides methods for iontophoretic transdermal delivery of non-peptidic polymeric agents, particularly non-peptidic polymeric cosmetic agents.

The present invention is largely based on the amphoteric feature of peptides, polypeptides and proteins. Being amphoteric, peptides, polypeptides and proteins can assume high positive charge or high negative charge if dissolved in solutions having lower or higher pH than their isoelectric pH. Thus, a cosmetic or pharmaceutical composition comprising as an active agent a peptide, polypeptide or protein and a pharmaceutically acceptable carrier, wherein the pharmaceutically acceptable carrier has a pH below or above the isoelectric pH of the peptide, polypeptide or protein enables increasing the positive or negative charge of the peptide, polypeptide or protein, and thereby facilitating efficient transdermal delivery of said peptide, polypeptide or protein by iontophoresis.

It is now disclosed for the first time that iontophoretic transdermal delivery of a non-peptidic polymeric cosmetic or therapeutic agent is improved when it is delivered with a peptide, polypeptide or protein, the non-peptidic polymeric cosmetic or therapeutic agent and the peptide, polypeptide or protein are present in a cosmetic or pharmaceutical composition comprising a pharmaceutically acceptable carrier having a pH below or above the isoelectric pH of the peptide, polypeptide or protein. It is now disclosed that placing at least one reservoir of an iontophoresis device in physical contact with the skin, the reservoir comprising a cosmetic or pharmaceutical composition comprising as active ingredients a non-peptidic polymeric cosmetic or therapeutic agent and a peptide, polypeptide or protein, and connecting anode and cathode electrodes of the iontophoretic device to a power supply, wherein the anode or cathode electrode is electrically connected to the reservoir greatly enhances the transdermal delivery of the cosmetic or therapeutic agent when delivered with the peptide, polypeptide or protein as compared to its delivery in the absence of said peptide, polypeptide or protein.

It is further disclosed that iontophoretic delivery of a non-peptidic polymeric cosmetic or therapeutic agent together with a peptide, polypeptide or protein through the skin is further improved if the skin is subjected to removal of the stratum corneum prior to iontophoresis.

According to a first aspect, the present invention provides a system for iontophoretic transdermal delivery of a non-peptidic polymeric cosmetic or therapeutic agent, the system comprises an iontophoresis device comprising at least one agent reservoir adapted for holding a cosmetic or pharmaceutical composition comprising as active ingredients a non-peptidic polymeric cosmetic or therapeutic agent and a peptide, polypeptide or protein and a pharmaceutically acceptable carrier, the pharmaceutically acceptable carrier has a pH of at least one pH unit below or above the isoelectric pH of the peptide, polypeptide or protein, thereby increasing the positive or negative charge of said peptide, polypeptide or protein.

According to some embodiments, the non-peptidic polymeric cosmetic or therapeutic agent is selected from the group consisting of polysaccharides, polynucleotides, and synthetic polymers. According to additional embodiments, the polysaccharide is selected from the group consisting of hyaluronic acid, polylactic acid, heparin, heparan sulphate, dermatan sulphate and chondroitin sulphate.

According to further embodiments, the peptide, polypeptide or protein is selected from the group consisting of insulin, proinsulin, follicle stimulating hormone, insulin like growth factor-1, insulin like growth factor-2, platelet derived growth factor, epidermal growth factor, fibroblast growth factors, nerve growth factor, colony stimulating factors, transforming growth factors, tumor necrosis factor, calcitonin, parathyroid hormone, growth hormone, bone morphogenic protein, erythropoietin, hemopoietic growth factors, luteinizing hormone, calcitonin, glucagons, clotting factors such as factor VIIIC, factor IX, tissue factor, and von Willebrand factor, anti-clotting factors such as Protein C, atrial natriuretic factor, lung surfactant, plasminogen activator, such as urokinase or tissue-type plasminogen activator, including human tissue-type plasminogen activator (t-PA), bombesin, thrombin, enkephalinase, a collagen domain, mullerian-inhibiting agent, relaxin A-chain, relaxin B-chain, prorelaxin, Dnase, inhibin, activin, vascular endothelial growth factor, receptors for hormones or growth factors, integrin, protein A or D, a neurotrophic factor such as bone-derived neurotrophic factor (BDNF), neurotrophin-3, -4, -5, or -6 (NT-3, NT-4, NT-5, or NT-6), immunotoxins, interferon such as interferon-alpha, -beta, and -gamma; colony stimulating factors (CSFs), interleukins (ILs) such as IL-1 to IL-10; surface membrane proteins, viral antigen such as a portion of the AIDS envelope, transport proteins, homing receptors, regulatory proteins, collagen, gelatin, fibrin, keratin, elastin, laminin, bovine serum albumin (BSA), polylysine, antibodies, glycoproteins, phosphoproteins, avidin-biotin complexes and lectins.

According to some embodiments, the pharmaceutically acceptable carrier has a pH of at least two pH units below or above the isoelectric pH of the peptide, polypeptide or protein. According to additional embodiments, the pharmaceutically acceptable carrier has a pH of at least three pH units below or above the isoelectric pH of the peptide, polypeptide or protein.

According to further embodiments, the cosmetic or pharmaceutical composition can further comprise a cosmetic or therapeutic agent.

According to some embodiments, the cosmetic agent is selected from the group consisting of xanthines, retinoids, α-hydroxy acids, β-hydroxy acids, α-2 adrenergic inhibitors, β-adrenergic agonists, aromatase inhibitors, anti-estrogens, hydroquinone, vitamins, kojic acid, corticosteroids, estrogens, isoflavonoids, cinnamic acid, benzoyl peroxide, tropolone, catechol, mercaptoamine, niacinamide, ferulic acid, azelaic acid, botulinum, colors, dermal fillers such as silicone and oil particles, derivatives and salts thereof.

According to additional embodiments, the therapeutic agent is selected from the group consisting of anti-infectives, analgesics, anesthetics, antiarthritic agents, antiasthmatic agents, anticonvulsants, anti-depressants, anti-diabetic agents, anti-diarrhea agents, anti-histamines, anti-inflammatory agents, anti-migraine agents, anti-motion sickness preparations, anti-neoplastics, anti-parkinsonism drugs, anti-pruritics, anti-psychotics, antipyretics, anti-spasmodics, anticholinergics, sympathomimetics, xanthine derivatives, calcium channel blockers, beta-blockers, anti-arrhythmics, anti-hypertensives, diuretics, vasodilators, central nervous system stimulants, cough suppressants, cold preparations, decongestants, diagnostics, hormones, hypnotics, immunosuppressives, muscle relaxants, parasympathomimetics, psychostimulants, sedatives tranquilizers, food supplements, derivatives and salts thereof.

According to a currently exemplary embodiment, the present invention provides a system for iontophoretic transdermal delivery of a non-peptidic polymeric cosmetic or therapeutic agent, the system comprises an iontophoresis device comprising two agent reservoirs, a first agent reservoir adapted for holding a first cosmetic or pharmaceutical composition comprising as active ingredients a non-peptidic polymeric cosmetic or therapeutic agent and a peptide, polypeptide or protein, and a first pharmaceutically acceptable carrier, wherein the first pharmaceutically acceptable carrier has a pH of at least one pH unit below the isoelectric pH of the protein, and a second agent reservoir adapted for holding a second cosmetic or pharmaceutical composition comprising as active ingredients the non-peptidic polymeric cosmetic agent and the peptide, polypeptide or protein, and a second pharmaceutically acceptable carrier, the second pharmaceutically acceptable carrier has a pH of at least one pH unit above the isoelectric pH of said peptide, polypeptide or protein. According to certain embodiments, the non-peptidic polymeric cosmetic agent is hyaluronic acid and the protein is bovine serum albumin or collagen.

It is to be understood that the present invention encompasses systems for iontophoretic transdermal delivery comprising two agent reservoirs, wherein a first agent reservoir adapted for holding a first pharmaceutical composition comprising a first non-peptidic polymeric cosmetic or therapeutic agent and a first peptide, polypeptide or protein, and wherein a second agent reservoir adapted for holding a second pharmaceutical composition comprising a second non-peptidic polymeric cosmetic or therapeutic agent and a second peptide, polypeptide or protein so long as the first pharmaceutical composition is positively charged and the second pharmaceutical composition is negatively charged. Thus, the present invention provides a system useful for simultaneous transdermal delivery of different non-peptidic polymeric cosmetic or therapeutic agents and different peptides, polypeptides or proteins.

According to another aspect, the present invention provides a system for iontophoretic transdermal delivery of a polypeptide comprising at least one hundred amino acid residues, the system comprises an iontophoresis device comprising at least one agent reservoir adapted for holding a cosmetic or pharmaceutical composition, wherein the cosmetic or pharmaceutical composition comprises as an active ingredient a polypeptide comprising at least one hundred amino acid residues, and a pharmaceutically acceptable carrier, wherein the pharmaceutically acceptable carrier has a pH of at least one pH unit below or above the isoelectric pH of said polypeptide, thereby increasing the positive or negative charge of the polypeptide. According to some embodiments, the polypeptide is selected from the group consisting of collagen, gelatin, fibrin, epidermal growth factors, keratin, elastin, laminin, and the like.

According to a currently exemplary embodiment, the present invention provides a system for iontophoretic transdermal delivery of a polypeptide, the system comprises an iontophoresis device comprising a first agent reservoir adapted for holding a first cosmetic or pharmaceutical composition comprising as an active ingredient a polypeptide comprising at least one hundred amino acid residues, and a first pharmaceutically acceptable carrier, wherein the first pharmaceutically acceptable carrier has a pH of at least one pH unit below the isoelectric pH of said polypeptide, and a second agent reservoir adapted for holding a second cosmetic or pharmaceutical composition comprising as an active ingredient the polypeptide and a second pharmaceutically acceptable carrier, wherein the second pharmaceutically acceptable carrier has a pH of at least one pH unit above the isoelectric pH of said polypeptide. According to a certain exemplary embodiment, the polypeptide is collagen.

According to a further aspect, the present invention provides a method for iontophoretic transdermal delivery of a non-peptidic polymeric cosmetic or therapeutic agent, the method comprising the steps:
a) removing the stratum corneum from a predetermined site on the skin of a subject;
b) providing a system for iontophoretic transdermal delivery comprising an iontophoresis device comprising at least one agent reservoir adapted for holding a cosmetic or pharmaceutical composition comprising as active ingredients a non-peptidic polymeric cosmetic or therapeutic agent and a peptide, polypeptide or protein, and a pharmaceutically acceptable carrier, wherein the pharmaceutically acceptable carrier has a pH of at least one pH unit below or above the isoelectric pH of the peptide, polypeptide or protein, thereby increasing the positive or negative charge of said peptide, polypeptide or protein;
c) placing said at least one agent reservoir adapted for holding the cosmetic or pharmaceutical composition of step (b) in contact with the site on the skin of the subject; and
d) delivering the non-peptidic polymeric cosmetic or therapeutic agent and the peptide, polypeptide or protein to the subject by iontophoresis.

According to some embodiments, removing the stratum corneum is performed by abrasion. According to additional embodiments, removing the stratum corneum by abrasion is selected from the group consisting of mechanical abrasion, thermal abrasion and chemical abrasion. According to further embodiments, removing the stratum corneum is performed by chemical microporation.

According to a currently exemplary embodiment, the present invention provides a method for iontophoretic transdermal delivery of a non-peptidic polymeric cosmetic or therapeutic agent comprising the steps:
a) removing the stratum corneum from a predetermined site on the skin of a subject;
b) providing a system for iontophoretic transdermal delivery comprising an iontophoresis device comprising a first agent reservoir adapted for holding a first cosmetic or pharmaceutical composition comprising as active ingredients a non-peptidic polymeric cosmetic or therapeutic agent and a peptide, polypeptide or protein, and a first pharmaceutically acceptable carrier, wherein the first pharmaceutically acceptable carrier has a pH of at least one pH unit below the isoelectric pH of the peptide, polypeptide or protein, and a second agent reservoir adapted for holding a second cosmetic or pharmaceutical composition comprising as active ingredients the non-peptidic polymeric cosmetic or therapeutic agent and the peptide, polypeptide or protein, and a second pharmaceutically acceptable carrier, wherein the second pharmaceutically acceptable carrier has a pH of at least one pH unit above the isoelectric pH of the peptide, polypeptide or protein;
c) placing the first and second agent reservoirs adapted for holding the cosmetic or pharmaceutical composition of step (b) in contact with the site on the skin of the subject; and
d) delivering the non-peptidic polymeric cosmetic or therapeutic agent and the peptide, polypeptide or protein to the subject by iontophoresis.

According to further embodiments, the cosmetic or pharmaceutical composition further comprises a cosmetic agent or therapeutic agent according to the principles of the present invention.

According to a further aspect, the present invention provides a method for iontophoretic transdermal delivery of a polypeptide comprising at least one hundred amino acid residues, the method comprises the steps:
a) removing the stratum corneum from a predetermined site on the skin of a subject;
b) providing a system for iontophoretic transdermal delivery comprising an iontophoresis device comprising at least one agent reservoir adapted for holding a cosmetic or pharmaceutical composition, wherein the cosmetic or pharmaceutical composition comprises as an active ingredient a polypeptide comprising at least one hundred amino acid residues and a pharmaceutically acceptable carrier, wherein the pharmaceutically acceptable carrier has a pH of at least one pH unit below or above the isoelectric pH of the polypeptide, thereby increasing the positive or negative charge of said polypeptide;
c) placing said at least one agent reservoir of step (b) in contact with the site on the skin of the subject, wherein the agent reservoir is electrically connected to an anode or cathode electrode; and
d) delivering the polypeptide to the subject by iontophoresis.

According to yet further embodiment, the present invention provides a method for iontophoretic transdermal delivery of a polypeptide comprising at least one hundred amino acid residues, the method comprises the steps:
a) removing the stratum corneum from a predetermined site on the skin of a subject;
b) providing a system for iontophoretic transdermal delivery comprising an iontophoresis device comprising a first agent reservoir adapted for holding a first cosmetic or pharmaceutical composition, wherein the first cosmetic or pharmaceutical composition comprises as an active ingredient a polypeptide comprising at least one hundred amino acid residues, and a first pharmaceutically acceptable carrier, wherein the first pharmaceutically acceptable carrier has a pH of at least one pH unit below the isoelectric pH of the polypeptide, and a second agent reservoir adapted for holding a second cosmetic or pharmaceutical composition comprising as an active ingredient the polypeptide, and a second pharmaceutically acceptable carrier, wherein the second pharmaceutically acceptable carrier has a pH of at least one pH unit above the isoelectric pH of said polypeptide;
c) placing the first and second agent reservoirs of step (b) in contact with the site on the skin of the subject; and
d) delivering the polypeptide to the subject by iontophoresis.

According to some embodiments, the methods of iontophoretic transdermal delivery of a non-peptidic polymeric cosmetic or therapeutic agent or a polypeptide comprising at least one hundred amino acid residues according to the principles of the present invention are useful for treating a disease or condition in a subject in need thereof. According to preferred embodiments, the methods of the present invention are useful for treating skin degenerative conditions including, but not limited to, cellulite, acne vulgaris, acne cystic, skin aging, skin wrinkles, hyper pigmentation, keratosis, skin blemish, dandruff, warts, photodamaged skin, chronic dermatoses, dermatitis, dryness, ichthyosis, viral skin infections, fungal skin infections, and bacterial skin infections. According to additional embodiments, the diseases or conditions to be treated by the methods of the present invention include, but are not limited to, diabetes, osteoporosis, cardiovascular diseases, inflammatory diseases, degenerative neurological diseases, degenerative muscle diseases, bacterial infections and impotence.

These and other embodiments of the present invention will be better understood in relation to the figures, description, examples and claims that follow.

### BRIEF DESCRIPTION OF THE FIGURES

FIGS. 1A-B are schematic representation of an iontophoretic delivery device without substances (FIG. 1A) or with substances S1 and S2 (FIG. 1B). B1 and B2 designate the anode and cathode, respectively

FIG. 2 is a schematic representation of an additional iontophoretic delivery device.

FIG. 3 is a graphic representation of iontophoretic delivery of collagen through pig ear skin piece.

FIG. 4 is a graphic representation of an additional experiment of iontophoretic delivery of collagen through pig ear skin piece.

FIG. 5 is a schematic representation of the iontophoretic device used for collagen delivery.

### DETAILED DESCRIPTION OF THE INVENTION

The terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

The term “transdermal”, as used herein, means the delivery of an active ingredient or agent into and/or through the skin.

The term “iontophoresis”, as used herein, refers generally to the delivery of an active ingredient or agent through the skin wherein the delivery is induced or aided by the application of electric current. As is known in the art, iontophoresis involves the electrically induced transport of charged ions.

A “peptide” refers to a polymer in which the monomers are amino acids linked together through amide bonds. Peptides are smaller than proteins, typically under 50 amino acid residues in total.

A “polypeptide” refers to a single polymer of amino acids, generally over 50 amino acids.

A “protein” as used herein refers to polymers of amino acids typically over 50 amino acids.

### Iontophoresis Device

As used herein, the term “iontophoresis device” refers generally to an electrically assisted device or apparatus suitable for the transdermal iontophoretic delivery of an agent to a subject. Such iontophoresis devices are well known in the art and are also referred to as “iontophoretic delivery devices”.

It should be understood that the methods of the invention are not limited to any particular iontophoresis device. Any known iontophoresis device or iontophoresis electrode may be utilized in the present invention so long as the active ingredient is iontophoretically delivered into or through the skin of a subject.

Iontophoresis devices useful in the present invention are described, for example, in the following U.S. patent documents, the disclosures of which are incorporated by reference as if fully set forth herein: U.S. Pat. Nos. 3,991,755; 4,141,359; 4,250,878; 4,398,545; 4,744,787; 4,747,819; 4,927,408; 5,125,894; 5,169,382; 5,203,768; 5,207,752; 5,236,412; 5,302,172; 5,310,404; 5,314,502; 5,320,598; 5,405,317; 5,464,387; 5,458,569; 5,935,598.

Typically, the iontophoresis delivery device used in the present invention comprises a power source for generation of an electrical current and two electrode assemblies that, when adhering to the skin of a subject, will pass a generated electrical current through the subject's skin. In the presence of the electrical current, the passage of an active ingredient from an agent reservoir through the skin is enhanced. As is appreciated by one of skill in the art of iontophoresis, the rate of transdermal delivery of an active ingredient in accordance with the present invention can be controlled by appropriate selection of a patch design, including the selection of the contents of the agent reservoir and electrolyte reservoir, the surface area of the patch, and by the strength of the generated electrical current.

In general, the iontophoresis device includes at least two electrode assemblies. Both of the electrode assemblies are disposed so as to be in intimate electrical contact with some portion of the skin of the body. The circuit of the device is completed by connection of the electrode assemblies to a source of electrical energy, for example, a battery, in conjunction with the electrode assembly contacts with the patient's skin.

In electrical terms, a first electrode assembly includes a positive electrode or “anode,” and a second electrode assembly includes a negative electrode or a “cathode.” According to some embodiments, the anode is electrically connected to an agent reservoir and the cathode is electrically connected to an electrolyte reservoir to complete the electrical circuit. According to other embodiments, the cathode is electrically connected to an agent reservoir and the anode is electrically connected to an electrolyte reservoir to complete the electrical circuit. According to additional embodiments, the anode and the cathode can be used to deliver an active agent of opposite charge, and thus the anode and cathode each is electrically connected to an agent reservoir. Under these conditions, both electrodes are considered to be active or donor electrodes: the anode is used to deliver a positively charged ionic agent into the body while the cathode is used to deliver a negatively charged ionic agent into the body.

It is to be understood that the electrode assembly comprising an agent reservoir is typically adapted to be placed in agent transmitting relation to the skin of the subject.

### A. The Electrodes

The electrodes or current distributing members can be constructed of any of a large variety of electrically conductive materials, including inert and sacrificial materials.

Inert conductive materials are those electrically conductive materials that, when employed in the iontophoretic devices of the invention, do not themselves undergo or participate in electrochemical reactions. Thus, an inert material distributes current without being eroded or depleted due to the distribution of the current, and conducts current through generation of hydronium ions (H₃O⁺) or hydroxyl ions (OH⁻) by, respectively, reduction or oxidation of water. Inert conductive materials typically include, for example, stainless steel, platinum, gold, and carbon or graphite.

Alternatively, the electrode can be constructed from a sacrificial conductive material. A material can be considered sacrificial if, when employed as an electrode in an iontophoretic device of the invention, the material is eroded or depleted due to its oxidation or reduction. Such erosion or depletion occurs when the materials and formulations used in the iontophoresis device enable a specific electrochemical reaction, such as when a silver electrode is used with a formulation containing chloride ions. In this situation, the current distributing member would not cause electrolysis of water, but would itself be oxidized or reduced.

Typically, for anodes, a sacrificial material would include an oxidizable metal such as silver, zinc, copper, aluminum, etc. In contrast to the hydroxyl and hydronium ions electrochemically generated via an inert material, the ions electrochemically generated via a sacrificial material would include metal cations resulting from oxidation of the metal. Metal/metal salt anodes can also be employed. In such cases, the metal would oxidize to metal ions, which would then be precipitated as an insoluble salt.

For cathodes, a sacrificial electrode can be constructed from any electrically conductive material provided an appropriate electrolyte formulation is provided. For example, a cathodic electrode can be constructed from a metal/metal salt material. A preferred cathodic material is a silver/silver halide material. In such embodiments, a metal halide salt is preferably employed as the electrolyte. In this case, the device would generate halide ions from the electrode as the metal is reduced electrochemically. Also, accompanying silver ions (Ag⁺) in a formulation would be reduced to silver metal (Ag(s)) and would deposit (plate) onto the electrode. In other embodiments, the cathode material can be an intercalation material, an amalgam, or other material that can take electrolyte cations such as sodium out of solution, below the reduction potential of water.

In addition, other materials can be used that permit the plating out of a metal from the appropriate electrolyte solution. Thus, metals such as silver, copper, zinc, and nickel, and other materials, such as carbon, can be employed when an appropriate metal salt such as silver nitrate or zinc sulfate is in solution in the electrolyte reservoir. While such materials may develop increased resistivity as metal plates out during use, they are not eroded or depleted during use as cathodic electrodes. They are therefore not strictly “sacrificial” in this context. Nonetheless, the term “sacrificial” encompasses such materials as it is intended to include materials that undergo physical and/or chemical changes during iontophoresis, such as to affect their function as measured by their lifetime or current carrying capacity, etc.

The electrode can take any form known in the art, such as the form of a patch, plate, foil layer, screen, wire, dispersion of conductive particles embedded in a conductive matrix, and the like. A variety of iontophoresis patch designs can be suitably used in the present invention as known in the art. For example, iontophoresis delivery devices have been developed in which the electrode assemblies, i.e., the donor and counter electrode assemblies have a “multi-laminate” construction (see, for example, U.S. Pat. Nos. 4,731,049 and 4,474,570).

The electrodes of the invention convey electrical current into at least one agent reservoir for the delivery of an ionized active ingredient into and/or through the skin of a subject. Typically, in iontophoresis devices, the two electrodes are arranged in electrical communication with a power supply. An electric signal is thus applied to the iontophoresis electrode pair from the power supply. Alternatively, the electrodes can be of a single polarity and the body is grounded by an additional electrode having an opposite potential and connected at a far location.

In typical transdermal iontophoresis system a low constant current, ranging from micro-Amps to several mAmps, is applied for prolonged periods of time ranging from micro seconds to days. Alternatively, low constant voltage, ranging from mVolts to several tens of volts is applied for prolonged periods of time ranging from micro seconds to days. The target amperage or voltage may also be achieved by a slow ramping up of the applied electric condition. Alternatively, starting from the target amperage or voltage, the electrical conditions may also be ramped down over time. Alternatively, consecutive pulses using the above electrical conditions are applied during the total duration of iontophoresis. Further alternatively, consecutive pulses of high voltage-high current for short periods of time such as for about 1 micro second to about 10 seconds are within the scope of the present invention.

According to some embodiments of the invention, after the iontophoresis device is placed on the patient's skin, a current in the range of approximately 50 μA-20 mA is applied over a time period that ranges from 1 micro second to 1 day.

According to alternative embodiments, after the iontophoresis device is placed on the patient's skin, a voltage in the range of approximately 0.5 V-20 V is applied over a time period that ranges from 1 micro second to 1 day.

According to additional embodiments of the invention, after the iontophoresis device is placed on the patient's skin, the target amperage or voltage is achieved by a slow ramping up of the applied electric condition.

According to alternative embodiments, starting from the target amperage or voltage, the electrical conditions are ramped down over time.

According to additional embodiments, consecutive pulses lasting from 1 micro second to 12 hours, preferably from 1 micro second to 10 seconds, using the above electrical conditions are applied during the total duration of iontophoresis.

### B. The Electrolyte Reservoir

In the iontophoresis device of the invention, an electrolyte reservoir is constructed to permit electrical communication with an electrode. Typically, electrical communication requires that electrons of the electrode be exchanged with ions in the electrolyte reservoir upon the application of electrical current. Such electrical communication is preferably not impeded to any excessive degree by any intervening material(s) used in the construction of the iontophoretic device. In other words, the resistivity of the interface between the electrode and the electrolyte reservoir is preferably low.

The electrolyte reservoir contains at least one electrolyte, i.e., an ionic or ionizable component that can act to conduct current toward or away from the electrode. Typically, the electrolyte comprises one or more mobile ions, the selection of which is dependent upon the desired application. Examples of suitable electrolytes include aqueous solutions of salts. A preferred electrolyte is an aqueous solution of sodium chloride (NaCl), having a concentration of less than 1 mole/liter (<1 M), more preferably at about physiological concentration. Other suitable electrolytes include salts of physiological ions including, but not limited to, potassium (K⁺), chloride (Cl⁻), and phosphate (PO₄³⁻). The salt and its concentration can be selected as desired for particular applications.

Other chemical species can be selected by the skilled artisan for inclusion in the electrolyte reservoir. Such other species include, without limitation, chelation agents (e.g., citrate ions, EDTA) surfactants (e.g., non-ionic, cationic, or anionic), buffers, ionic excipients, osmolarity adjusters (e.g., polyethylene glycols, sugars), ionic antibiotics, penetration enhancers (e.g., alkanols), stabilizers, enzyme inhibitors, preservatives, thickening agents (e.g., acrylic acids, cellulose resins, clays, polyoxyethylenes), and the like. Inclusion of such species is made to selectively control or modulate the function of the electrolyte reservoir in particular circumstances.

Alternatively, the electrolyte can comprise a material that is itself relatively immobile in the absence of an electric field, but that acts to deliver mobile ions in the presence of an electric field. In the latter case, the electrolyte can more properly be termed an “ion source”. Examples of ion sources according to the invention include polyelectrolytes, ion exchange membranes and resins, non-ionic buffers that become ionic upon pH change, and other known ion sources.

### C. The Agent Reservoir

The iontophoresis device of the invention further includes an agent reservoir in the electrode assembly, which contains the active ingredient to be delivered. Preferably, the active ingredient is present as an ionized or ionizable form. The agent reservoir must be capable of ionic communication with the skin, which means that the boundary between the agent reservoir and the skin must be permeable to the complex (and may also be permeable to other ions), as the current is carried by ions traversing across the boundary. The agent reservoir is also in electrical communication with the anode or the cathode of the iontophoresis device.

The construction of the agent reservoir must be consistent with the requirements for ionic communication with the skin and electrical communication with the electrode. Accordingly, the structure of the agent reservoir would vary, depending upon the desired application. The agent can include a liquid, semi-liquid, semi-solid, or solid material.

The matrix of the agent reservoir can be of any material adapted to absorb and hold a sufficient quantity of liquid therein in order to permit transport of the agent there through by iontophoresis. For example, gauzes made of cotton or other absorbent fabrics as well as pads and sponges, both natural and synthetic, may be used. Most preferably, the matrix of the agent reservoir is composed, at least in part, of a hydrophilic polymer material. Both natural and synthetic hydrophilic polymers may be used. Suitable hydrophilic polymers include polyvinylpyrrolidones, polyvinyl alcohol, polyethylene oxides such as Polyox® manufactured by Union Carbide Corp.; Carbopol® manufactured by BF Goodrich of Akron, Ohio; blends of polyoxyethylene or polyethylene glycols with polyacrylic acid such as Polyox® blended with Carbopol®, polyacrylamide, Klucel®, cross-linked dextran such as Sephadex (Pharmacia Fine Chemicals, AB, Uppsala, Sweden), Water Lock® (Grain Processing Corp., Muscatine, Iowa) which is a starch-graft-poly(sodium acrylate-co-acrylamide) polymer, cellulose derivatives such as hydroxyethyl cellulose, hydroxypropylmethylcellulose, low-substituted hydroxypropylcellulose, and cross-linked Na-carboxymethylcellulose such as Ac-Di-Sol (FMC Corp., Philadelphia, Pa.) hydrogels such as polyhydroxyethyl methacrylate (National Patent Development Corp.), natural gums, chitosan, pectin, starch, guar gum, locust bean gum, and the like, along with blends thereof.

Hydrophilic polymers can be desired since water is the preferred solvent for ionizing many agents, and hydrophilic polymer components of the agent reservoir can be hydrated in situ while attached to the body by absorbing water from the skin through transepidermal water loss or sweat. Once hydrated, the device begins to deliver ionized agent to the body. This enables the agent reservoir to be manufactured in a dry state, giving the device a longer shelf life. Hydrogels are particularly favored for use as the agent reservoir matrix in iontophoresis delivery devices, in part due to their high equilibrium water content and their ability to quickly absorb water. In addition, hydrogels tend to have good biocompatibility with the skin. The pharmaceutical or cosmetic composition comprising an active agent and a pharmaceutically acceptable carrier may be added to the reservoir matrix either at the time of manufacture or in the form of solutions at the time of use of the device. For example, when the pharmaceutical or cosmetic composition comprising an active agent and a pharmaceutically acceptable carrier is added to the reservoir matrix at the time of manufacture of the device, blending of the pharmaceutical or cosmetic composition with the reservoir matrix components can be accomplished mechanically either by milling, extrusion, or hot-melt mixing. The resulting dry state reservoirs may then be prepared by solvent casting, extrusion or by melt-processing, for example. In addition to the active agent and the pharmaceutically acceptable carrier, the reservoirs may also contain other conventional excipients such as chelation agents (e.g., citrate ions, EDTA), surfactants (e.g., non-ionic, cationic, or anionic), buffers, ionic excipients, osmolarity adjusters (e.g., polyethylene glycols, sugars), ionic antibiotics, penetration enhancers (e.g., alkanols), stabilizers, enzyme inhibitors, preservatives, thickening agents (e.g., acrylic acids, cellulosic resins, clays, polyoxyethylenes), and the like.

Alternatively, the reservoirs may be manufactured with no pharmaceutical or cosmetic composition. In such a case, the pharmaceutical or cosmetic composition comprising an active ingredient and a pharmaceutically acceptable carrier can be added to the reservoirs by adding a solution of the pharmaceutical or cosmetic composition to the appropriate reservoir matrix at the time of use.

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the active ingredient is administered. Suitable carriers are pH buffers such as acetates, phosphates, citrates, carbonates and the like.

It is desirable that the ion of the pharmaceutically acceptable carrier should have low ionic mobility. The limiting ionic mobility of this ion is preferably no greater that 1×10⁻⁴ cm²/volt-sec. It is to be understood that according to the principles of the present invention, iontophoretic delivery of an active ingredient is obtained by designing pharmaceutically acceptable carriers having specific pH so that the active ingredient within the agent reservoir will have a high positive or high negative charge.

### D. Protective Backing

The iontophoresis device of the invention can also include a suitable backing film positioned on top of the agent reservoir. The backing film provides protection against contamination and damage to the electrode, if present, and the agent reservoir of the device. The backing film can also serve to maintain constant pressure on the reservoir so that the agent is continuously delivered to the skin surface.

### E. Release Liner

The iontophoresis device of the invention optionally includes a release liner that can be affixed to the underside of the agent reservoir by an adhesive. The release liner protects the surface of the agent reservoir that contacts the skin from contamination and damage when the device is not in use. When the device is ready for use, the release liner can be peeled off to expose the skin-contacting surface of the agent reservoir for application of the device to a patient.

### Active Ingredients

The active ingredient that can be iontophoretically delivered by the methods of the present invention is a non-peptidic polymeric cosmetic agent, a non-peptidic polymeric therapeutic agent, a peptide, a polypeptide, and a protein.

The non-peptidic cosmetic or therapeutic agents include, but are not limited to, polysaccharides, polyions, polymeric particles, polynucleotides, and phospholipids. Polysaccharides include, but are not limited to, hyaluronic acid and polylactic acid; Polyions include, but are not limited to, polyphosphates, polysulphates, and the like. Polymeric particles include, but are not limited to, polyurethane polymers, polyurea polymers, acrylic polymers, vinyl polymers, polyester-polyurethane polymers, polyether-polyurethane polymers or mixtures thereof (see, for example, U.S. Pat. No. 5,650,159 and references therein, incorporated by reference as if fully set forth herein).

Examples of peptides, polypeptides or proteins that can be delivered by the methods of the present invention include, but are not limited to, collagen, BSA, gelatin, fibrin, keratin, elastin, laminin, antibodies, polylysine, glycoproteins, phosphoproteins, and avidin-biotin complexes. Preferably, the peptide, polypeptide or protein can exert a therapeutic or cosmetic activity. However, the present invention encompasses inert peptides, polypeptides or proteins. The term “inert” peptides, polypeptides or proteins refers to peptides, polypeptides or proteins that do not exert any therapeutic or cosmetic activity. In an exemplary embodiment, the protein is bovine serum albumin (BSA).

The present invention provides pharmaceutical or cosmetic compositions which further comprise a therapeutic or cosmetic agent.

Therapeutic agents that can be delivered according to the principles of the present invention include, but are not limited to, anti-infectives, analgesics, anesthetics, antiarthritic agents, antiasthmatic agents, anticonvulsants, anti-depressants, anti-diabetic agents, anti-diarrhea agents, anti-histamines, anti-inflammatory agents, anti-migraine agents, anti-motion sickness preparations, anti-neoplastics, anti-parkinsonism drugs, anti-pruritics, anti-psychotics, antipyretics, anti-spasmodics, anticholinergics, sympathomimetics, xanthine derivatives, calcium channel blockers, beta-blockers, anti-arrhythmics, anti-hypertensives, diuretics, vasodilators, central nervous system stimulants, cough suppressants, cold preparations, decongestants, diagnostics, hormones, hypnotics, immunosuppressives, muscle relaxants, parasympathomimetics, psychostimulants, sedatives, tranquilizers, and food supplements.

Cosmetic agents that can be delivered by the device of the present invention include, but are not limited to, vitamins such as vitamin A, B, D, ascorbic acid or tocopherol; colors; dermal fillers such as silicone or oil particles; xanthines; retinoids; α-hydroxy acids; p-hydroxy acids; α-2 adrenergic inhibitors; β-adrenergic agonists; aromatase inhibitors; anti-estrogens; hydroquinone; kojic acid; corticosteroids; estrogens; isoflavonoids; cinnamic acid; benzoyl peroxide; tropolone; catechol; mercaptoamine; niacinamide; ferulic acid; azelaic acid; and botulinum.

It is to be understood that iontophoretic delivery of a non-peptidic cosmetic or therapeutic agent together with a peptide, polypeptide or protein when both the non-peptidic polymeric cosmetic or therapeutic agent and the peptide, polypeptide or protein are dissolved in a pharmaceutically acceptable carrier having a pH of at least one pH unit below or above the isoelectric pH of the peptide, polypeptide or protein improves the transdermal delivery of the non-peptidic polymeric cosmetic or therapeutic agent if compared to the delivery of said agent when it is not delivered with the peptide, polypeptide or protein. It is to be appreciated that any amphoteric polymer can be used to deliver a non-peptidic polymeric cosmetic or therapeutic agent according to the principles of the present invention. Thus, an amphoteric polymer selected from the group consisting of polynucleotides, phospholipids, polyions and polymeric particles, can be included with a non-peptidic polymeric cosmetic or therapeutic agent in a pharmaceutical composition to be delivered iontophoretically. Typically, the pH of the pharmaceutically acceptable carrier is at least one pH unit below or above the isoelectric pH of the peptide, polypeptide or protein. Yet, the present invention encompasses pharmaceutically acceptable carriers having a pH of at least two or at least three pH units below or above the isoelectric pH of the peptide, polypeptide or protein.

The pharmaceutical or cosmetic composition of the present invention can comprise the peptide, polypeptide or protein and the non-peptidic polymeric therapeutic or cosmetic agent as a complex without the addition of a chemical cross-linking agent. Alternatively, the peptide, polypeptide or protein and the non-peptidic polymeric therapeutic or cosmetic agent can be coupled by a chemical cross-linking agent.

Chemical cross-linking agents and methods for coupling are well known in the art. For example, aldehydes such as glutaraldehyde or formaldehyde, potassium periodate/sodium borohydride, carbodiimide and 2-chloro-1-methyl-pyridinium iodide (CPMI) are useful agents for coupling (see, for example, Young et al. J. Biomaterials Sci. Polymer Edn. 15: 767-780, 2004). Alternatively, coupling can be performed by introducing avidin and biotin; introducing of sulfhydryl residues (thiolation); introducing of carboxylate groups; introducing of primary amine groups; introducing of aldehyde residues; introducing of hydrazide functional groups. Alternatively, coupling can be performed through amine-reactive chemical reactions; thiol-reactive chemical reactions; carboxylate-reactive chemical reactions; hydroxyl-reactive chemical reactions; aldehyde- and ketone-reactive reactions; active hydrogen reactive chemical reactions; NHS ester-mediated hapten-carrier conjugation; NHS ester-maleimide heterobifunctional cross-linker mediated hapten-carrier conjugation; active-hydrogen mediated hapten-carrier conjugation; diazonium and Mannich conjugation; glutaraldehyde-mediated hapten-carrier conjugation; reductive-amination-mediated hapten-carrier conjugation. It is to be understood that the coupling of a peptide, polypeptide or protein to a non-peptidic polymeric therapeutic or cosmetic agent should not interfere, inhibit or reduce the therapeutic or cosmetic properties of the polymeric therapeutic or cosmetic agent.

### Methods of Use

The present invention provides methods for iontophoretic transdermal delivery of a non-peptidic polymeric cosmetic or therapeutic agent or of a polypeptide comprising at least hundred amino acid residues. The methods of the present invention are useful for treating or preventing a disease or condition in a subject in need thereof. According to some embodiments, the methods of the present invention are useful for treating or preventing diabetes, osteoporosis, cardiovascular diseases, inflammatory diseases, muscular degenerative diseases, neuronal degenerative diseases, bacterial infections and impotence.

According to some preferred embodiments, the methods of the present invention are useful for treating a degenerative skin condition in a subject. The term “treating” when used in reference to degenerative skin conditions further includes “preventing” or “inhibiting” formation of any symptoms of a degenerative skin condition. The dermatological treatment methods can also be used to promote healing of skin that has been damaged, either by trauma or by surgery.

The methods of the present invention can be practiced upon any part of the body where a degenerative skin condition (i.e., aging) appears. In humans, the most commonly treated areas of the body are face, hand, arm, neck, chest, or leg. The term “degenerative skin conditions” is used broadly herein, and refers to such symptoms as flabby or sagging skin as well as wrinkles, age spots, actinic damage caused by UV radiation, scars, laugh lines, stretch marks, acne scarring, subcutaneous atrophy, hypoplasia of the lips, and the like. Thus, degenerative skin conditions can result from either natural causes (such as aging), environmental causes (such as pollution and UV exposure), or such causes as poor diet. Disease conditions which inhibit endogenous production of collagen and/or oxy-radical scavengers, such as Vitamin C, or disrupt other natural processes that contribute to a healthy, more roseate, elastic skin can also contribute to degenerative skin conditions as the term is used herein.

The target for treatment of degenerative skin conditions or for cosmetic purposes is the dermis layer.

As used herein, the term “subject” refers to any animal. It is envisioned that the methods for iontophoretically delivering of a non-peptidic polymeric cosmetic or therapeutic agent or of a polypeptide comprising at least one hundred amino acid residues within a pharmaceutical composition can be performed on any animal. Preferably, the subject is a human.

It is specifically contemplated that a non-peptidic polymeric cosmetic or therapeutic agent or a polypeptide comprising at least one hundred amino acid residues administered locally may function systemically as well.

As used herein, the term “local” when used in reference to a non-peptidic polymeric therapeutic or cosmetic agent or to a polypeptide comprising at least one hundred amino acid residues, refers to its function in a particular region. Thus, a polypeptide or a non-peptidic polymeric therapeutic or cosmetic agent topically introduced into the skin are believed to exert their therapeutic or cosmetic activity function within the skin. Nevertheless, the skilled artisan will recognize that some topically introduced active ingredients may have a systemic effect or function, such that after topically introducing the active ingredient into the skin, the active ingredient is distributed to other areas of the subject thereby producing or contributing to treating degenerative skin conditions by acting at a site other than the skin. As used herein, the term “systemic” when used in reference to a peptide, polypeptide or protein or a therapeutic or cosmetic agent, means that the active ingredient functions outside the skin.

In accordance with the invention, an “effective amount” of a polypeptide or a non-peptidic polymeric therapeutic or cosmetic agent is an amount effective to produce a desired therapeutic and/or cosmetic effect, such as preventing or inhibiting a disease or condition in a subject, such as for example, a degenerative skin condition, or promoting healing such as skin healing in a subject in need thereof. An “effective amount” should not be so large as to cause excessive adverse side effects, such as skin irritation, burning, cytotoxicity, or tissue damage. The amount required for therapeutic or cosmetic treatment will vary from subject to subject, depending on the type of formulation, the species, age, and general condition of the subject (physiological and psychological), the severity of the condition being treated (e.g., chronic vs. acute), and the anatomical region of the skin being treated.

Thus, although it is not possible to specify an exact “effective amount”, an appropriate “effective” amount in any individual case may be determined by any method known in the art. For example, by using visual inspection to determine reduction in the number and/or depth of wrinkles, reduction in the amount and/or prominence of age spots, improvement in skin color (i.e., more roseate coloring) or by measuring certain skin parameters in response to various amounts of the cosmetic agent, one can readily determine an effective amount of the cosmetic agent. Skin parameters can include an increase in skin elasticity, an increase in blood supply, a reduced level of free oxygen radicals or enhanced collagen production in the treated skin region as compared with a comparable untreated skin region. The amount can be adjusted by the subject or, in the event of any complication, by a physician.

The methods of the present invention comprise steps that enhance the permeability of the stratum corneum of the skin, such as application of a permeation enhancer, electric dermabrasion or microdermabrasion, and the like. As used herein, the term “permeation enhancer” refers to any action (e.g., mechanical, physical, chemical) or any composition that can increase the topical delivery of a peptide, polypeptide or protein and a non-peptidic polymeric therapeutic or cosmetic agent into the skin. Permeation enhancer compositions that increase skin permeability include, for example, alcohols (e.g., methanol), alkyl methyl sulfoxides (e.g., DMSO), pyrrolidones (e.g., 2-pyrrolidone), surfactants, urea, glycerol monolaurate, polyethylene glycol monolaurate, glycerol monolaurate, docainehydrochloride, hydrocortisone, menthol, methyl salicylate, and the like. Examples of mechanical and physical actions that can be performed before the iontophoretic delivery of an active ingredient according to the principles of the present invention include, but are not limited to, application of pressure to the skin by rubbing or stroking the skin, by applying vibration such as ultrasound vibration, by heating the skin, by radiating the skin, for example by radio-frequency radiation, by cooling the skin, and the like.

Having now generally described the invention, the same will be more readily understood through reference to the following examples, which are provided by way of illustration and are not intended to be limiting of the present invention.

### Example 1

### Iontophoretic Delivery of Collagen in Intact Skin

Measurements were performed on collagen delivery through a 1.5 mm thick pig ear skin. The skin piece was obtained by slicing with scalpel a 1.5 mm thick section of a pig ear and fixed it over a 2 cm diameter laboratory vial. The vial was filled with a 50 ml solution of 0.05% NaCl in water to contact with the skin piece.

The experiment was performed by measuring the amount of collagen transmitted through the skin piece into the vial's solution by iontophoresis.

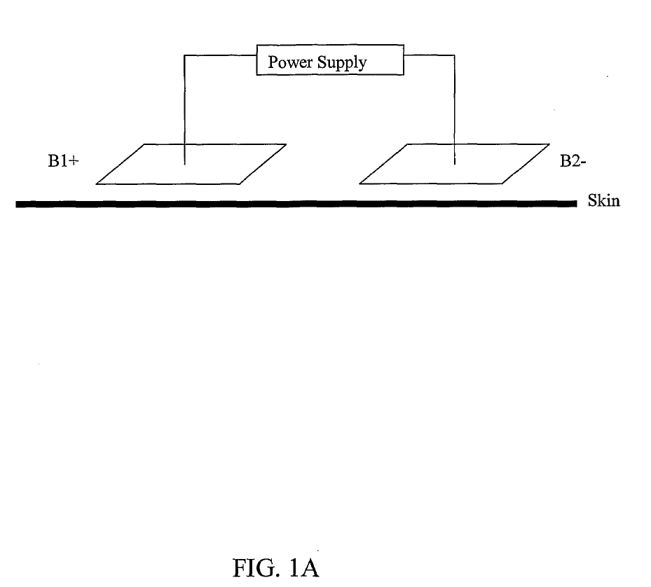

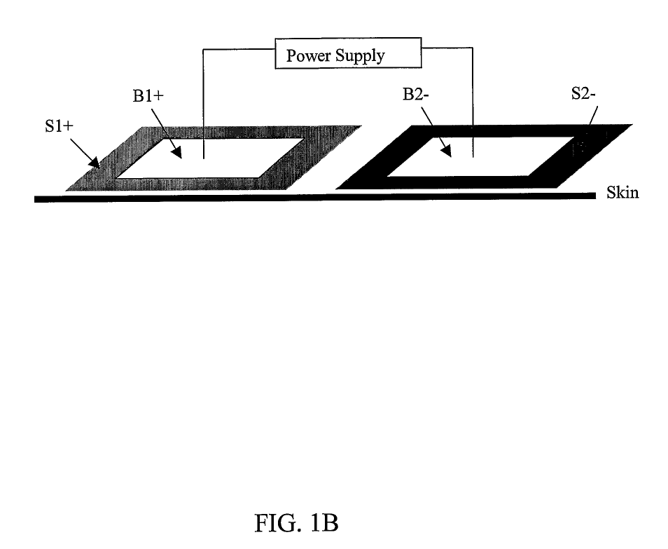

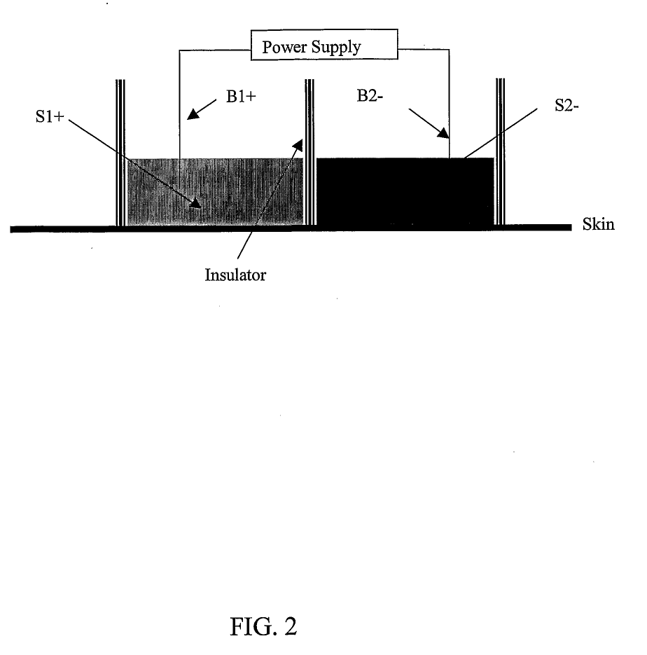

The experiments were performed on untreated skin using prototypes iontophoresis devices as shown in FIGS. 1 and 2.

Collagen (100 mg/ml; Sigma) was dissolved in 10 mM sodium phosphate pH 8 containing 150 mM NaCl or in 10 mM sodium phosphate pH 8 containing 150 mM NaCl to which 10 mM glutamic acid solution was added (0.2 ml of glutamic acid were added to 1 ml of collagen to yield pH 4.0), and the glutamic acid solution was incubated at room temperature for 1 hour. Thus, positively charged collagen was driven into the skin by the positive electrode and negatively charged collagen was driven by the negative electrode. The amount of collagen transmitted through the skin was measured by the Bradford assay (Bradford M. M. Anal Biochem. 72, 248-254, 1976).

The experiments were performed at a 15V DC applied voltage and at a current of 0.1 mA for 30 min.

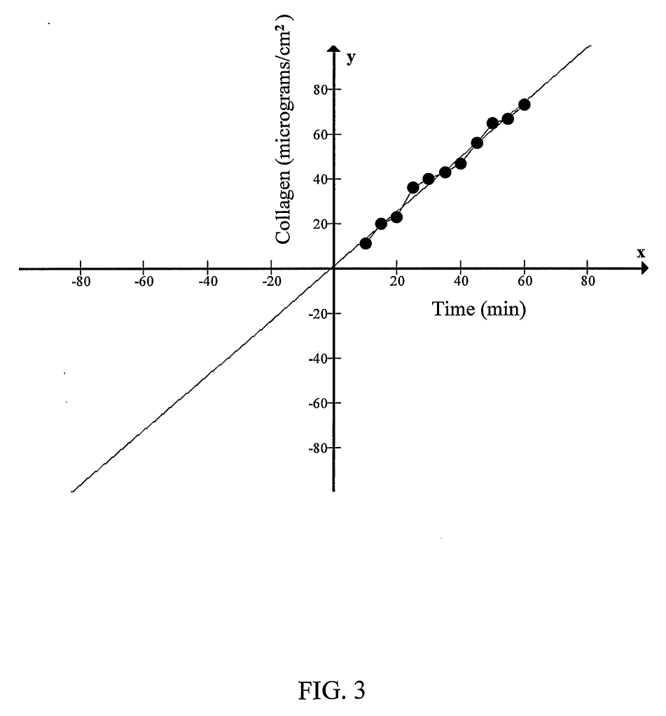

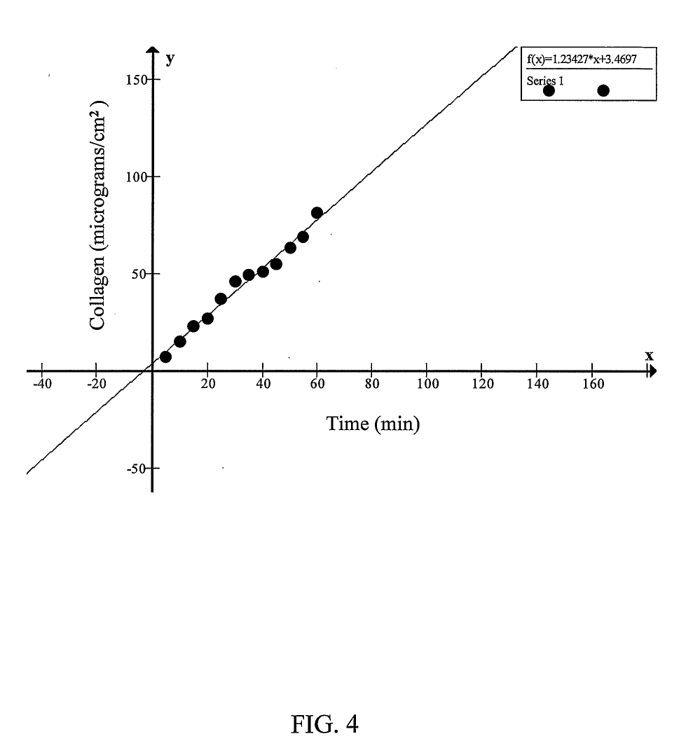

The amount of collagen collected in 5 min intervals was measured by the Bradford reagent. As shown in FIGS. 3 and 4, collagen was iontophoretically transmitted through the piece of pig ear skin into the solution.

### Example 2

### Iontophoretic Delivery of Collagen in Treated Skin

Pig ear skin piece was treated by chemical microporation using Silver Nitrite and then iontophoresis of collagen was performed as follows:

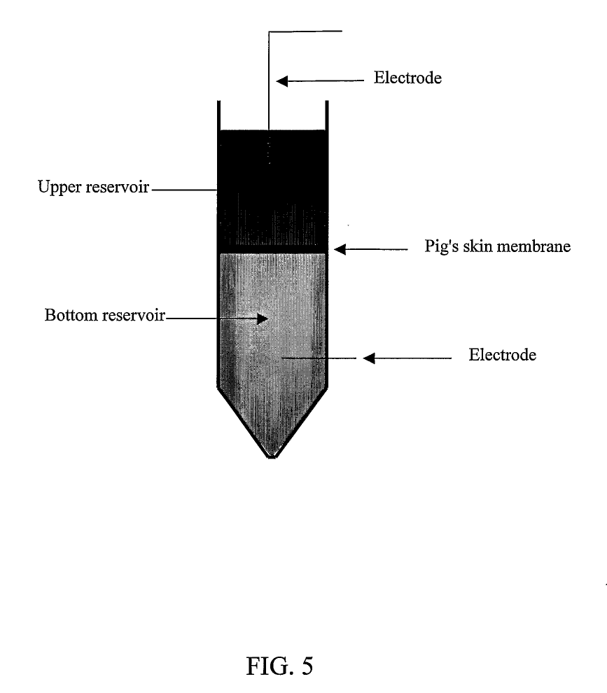

Twenty ml reservoir was mounted over the skin piece allowing its wetting (FIG. 5). Iontophoretic transmission experiment using the same solutions as described in Example 1 herein above was performed on each sample. Electrodes were placed in the upper and lower reservoirs. At the end of the experiment the amount of collagen transmitted through the skin was measured by the Bradford assay (Bradford M. M. Anal Biochem. 72, 248-254, 1976).
The iontophoresis was performed under the following conditions:
1. Distance between electrodes (upper and lower reservoir)—2 cm;

### 2. Voltage—15V;

### 3. Duration—1 h.

The results obtained are as follows:

| Initial amount (mg) | Transmitted amount (mg) | Transmitted amount (%) |
|---|---|---|
| 0.1 | 0.04 | 40 |
| 0.5 | 0.3 | 60 |
| 1.0 | 0.48 | 48 |
| 10.0 | 5.6 | 56 |
| 20.0 | 11.0 | 55 |

### Example 3

### Iontophoretic Delivery of Hyaluronic Acid in Treated Skin

Bovine serum albumin (BSA; Sigma) was dissolved in 10 mM sodium phosphate pH 7.2 containing 0.15 M NaCl to yield BSA concentration of 10 mg/ml. Hyaluronic acid suspension (1 mg; Q-Med) was incubated with 0.1 ml of the BSA solution at 40° C. In addition, 0.1 ml glutamic acid (10 mM) was added to another sample of the hyaluronic acid-BSA solution to yield pH 4.0. Piece of a pig ear skin was exposed to chemical microporation using Silver Nitrite and then iontophoretic delivery of the complex was performed as described in Example 1 herein above.

Hyaluronic acid transmitted through the pig ear was stained by complexation with a copper phtalocyanin dye and measured by the Bradford reagent.

The results obtained are as follows:

| Initial amount (mg) | Transmitted amount (mg) | Transmitted amount (%) |
|---|---|---|
| 0.1 | 0.46 | 46 |
| 0.05 | 0.030 | 60 |
| 0.01 | 0.007 | 70 |

It will be appreciated by persons skilled in the art that the present invention is not limited by what has been particularly shown and described herein above. Rather the scope of the invention is defined by the claims that follow.

1.-27. (canceled)

28. A system for iontophoretic transdermal delivery of a non-peptidic cosmetic or therapeutic agent comprising an iontophoresis device comprising at least one agent reservoir adapted for holding a cosmetic or pharmaceutical composition, wherein the cosmetic or pharmaceutical composition comprising as active ingredients a non-peptidic polymeric cosmetic or therapeutic agent and a peptide, polypeptide or protein, and a pharmaceutically acceptable carrier, wherein the pharmaceutically acceptable carrier has a pH of at least one pH unit below or above the isoelectric pH of the peptide, polypeptide or protein, thereby increasing the positive or negative charge of the peptide, polypeptide or protein.

29. The system according to claim 28, wherein the iontophoresis device comprises a first agent reservoir adapted for holding a first cosmetic or pharmaceutical composition comprising as active ingredients a non-peptidic polymeric cosmetic or therapeutic agent and a peptide, polypeptide or protein, and a first pharmaceutically acceptable carrier, wherein the first pharmaceutically acceptable carrier has a pH of at least one pH unit below the isoelectric pH of the peptide, polypeptide or protein, and a second agent reservoir adapted for holding a second cosmetic or pharmaceutical composition comprising as active ingredients the non-peptidic polymeric cosmetic or therapeutic agent and the peptide, polypeptide or protein, and a second pharmaceutically acceptable carrier, wherein the second pharmaceutically acceptable carrier has a pH of at least one pH unit above the isoelectric pH of the peptide, polypeptide or protein.

30. The system according to claim 28, wherein the non-peptidic polymeric cosmetic or therapeutic agent is selected from the group consisting of polysaccharides, polynucleotides, and synthetic polymers.

31. The system according to claim 30, wherein the polysaccharide is selected from the group consisting of hyaluronic acid, polylactic acid, heparin, heparan sulphate, dermatan sulphate and chondroitin sulphate.

32. The system according to claim 28, wherein the peptide, polypeptide or protein is selected from the group consisting of insulin, proinsulin, follicle stimulating hormone, insulin like growth factor-1, insulin like growth factor-2, platelet derived growth factor, epidermal growth factor, fibroblast growth factors, nerve growth factor, colony stimulating factors, transforming growth factors, tumor necrosis factor, calcitonin, parathyroid hormone, growth hormone, bone morphogenic protein, erythropoietin, hemopoietic growth factors, luteinizing hormone, calcitonin, glucagons, clotting factors such as factor VIIIC, factor IX, tissue factor, and von Willebrand factor, anti-clotting factors such as Protein C, atrial natriuretic factor, lung surfactant, plasminogen activator, such as urokinase or tissue-type plasminogen activator, including human tissue-type plasminogen activator (t-PA), bombesin, thrombin, enkephalinase, a collagen domain, mullerian-inhibiting agent, relaxin A-chain, relaxin B-chain, prorelaxin, Dnase, inhibin, activin, vascular endothelial growth factor, receptors for hormones or growth factors, integrin, protein A or D, a neurotrophic factor such as bone-derived neurotrophic factor (BDNF), neurotrophin-3, -4, -5, or -6 (NT-3, NT-4, NT-5, or NT-6), immunotoxins, interferon such as interferon-alpha, -beta, and -gamma; colony stimulating factors (CSFs), interleukins (ILs) such as IL-1 to IL-10; surface membrane proteins, viral antigen such as a portion of the AIDS envelope, transport proteins, homing receptors, regulatory proteins, collagen, gelatin, fibrin, keratin, elastin, laminin, bovine serum albumin (BSA), polylysine, antibodies, glycoproteins, phosphoproteins, avidin-biotin complexes and lectins.

33. The system according to claim 28, wherein the pharmaceutically acceptable carrier has a pH of at least two pH units below or above the isoelectric pH of the peptide, polypeptide or protein.

34. The system according to claim 28, wherein the pharmaceutically acceptable carrier has a pH of at least three pH units below or above the isoelectric pH of the peptide, polypeptide or protein.

35. The system according to claim 28, wherein the pharmaceutical or cosmetic composition further comprises an additional therapeutic or cosmetic agent.

36. The system according to claim 35, wherein the therapeutic agent is selected from the group consisting of anti-infectives, analgesics, anesthetics, antiarthritic agents, antiasthmatic agents, anticonvulsants, anti-depressants, anti-diabetic agents, anti-diarrhea agents, anti-histamines, anti-inflammatory agents, anti-migraine agents, anti-motion sickness preparations, anti-neoplastics, anti-parkinsonism drugs, anti-pruritics, anti-psychotics, antipyretics, anti-spasmodics, anticholinergics, sympathomimetics, xanthine derivatives, calcium channel blockers, beta-blockers, anti-arrhythmics, anti-hypertensives, diuretics, vasodilators, central nervous system stimulants, cough suppressants, cold preparations, decongestants, diagnostics, hormones, hypnotics, immunosuppressives, muscle relaxants, parasympathomimetics, psychostimulants, sedatives tranquilizers, food supplements, derivatives and salts thereof.

37. The system according to claim 35, wherein the cosmetic agent is selected from the group consisting of polysaccharides, xanthines, retinoids, α-hydroxy acids, β-hydroxy acids, α-2 adrenergic inhibitors, β-adrenergic agonists, aromatase inhibitors, anti-estrogens, hydroquinone, vitamins, kojic acid, corticosteroids, estrogens, isoflavonoids, cinnamic acid, benzoyl peroxide, tropolone, catechol, mercaptoamine, niacinamide, ferulic acid, azelaic acid, botulinum, colors, dermal fillers, derivatives and salts thereof.

38. The system according to claim 28, wherein the cosmetic composition comprises hyaluronic acid and BSA.

39. The system according to claim 28, wherein the cosmetic composition comprises hyaluronic acid and collagen.

40. A system for iontophoretic transdermal delivery of a polypeptide comprising at least one hundred amino acid residues, the system comprises an iontophoresis device comprising at least one agent reservoir adapted for holding a cosmetic or pharmaceutical composition, wherein the cosmetic or pharmaceutical composition comprises as an active ingredient a polypeptide comprising at least one hundred amino acid residues, and a pharmaceutically acceptable carrier, wherein the pharmaceutically acceptable carrier has a pH of at least one pH unit below or above the isoelectric pH of the polypeptide, thereby increasing the positive or negative charge of said polypeptide.

41. The system according to claim 40, wherein the iontophoresis device comprises a first agent reservoir adapted for holding a first cosmetic or pharmaceutical composition, wherein the first cosmetic or pharmaceutical composition comprises as an active ingredient a polypeptide comprising at least one hundred amino acid residues, and a pharmaceutically acceptable carrier, wherein the pharmaceutically acceptable carrier has a pH of at least one pH unit below the isoelectric pH of the polypeptide, and a second agent reservoir adapted for holding a second cosmetic or pharmaceutical composition, wherein the second cosmetic or pharmaceutical composition comprises as an active ingredient the polypeptide, and a second pharmaceutically acceptable carrier, wherein the second pharmaceutically acceptable carrier has a pH of at least one pH unit above the isoelectric pH of the polypeptide.

42. A method for iontophoretic transdermal delivery of a non-peptidic polymeric cosmetic or therapeutic agent, which comprises:
removing the stratum corneum from a predetermined site on the skin of a subject;
providing a system for iontophoretic transdermal delivery comprising an iontophoresis device comprising at least one agent reservoir adapted for holding a pharmaceutical or cosmetic composition, wherein the pharmaceutical or cosmetic composition comprises as active ingredients a non-peptidic polymeric cosmetic or therapeutic agent and a peptide, polypeptide or protein, and a pharmaceutically acceptable carrier, wherein the pharmaceutically acceptable carrier has a pH of at least one pH unit below or above the isoelectric pH of the peptide, polypeptide or protein, thereby increasing the positive or negative charge of said peptide, polypeptide or protein;
placing the at least one agent reservoir in contact with the site on the skin of the subject; and
delivering the non-peptidic polymeric cosmetic or therapeutic agent and the peptide, polypeptide or protein to the subject by iontophoresis.

43. The method according to claim 42, wherein the iontophoresis device comprises a first agent reservoir adapted for holding a first cosmetic or pharmaceutical composition comprising as an active ingredient said peptide, polypeptide, or protein and a first pharmaceutically acceptable carrier, wherein the first pharmaceutically acceptable carrier has a pH of at least one pH unit below the isoelectric pH of the peptide, polypeptide, or protein and a second agent reservoir adapted for holding a second cosmetic or pharmaceutical composition comprising as an active ingredient the peptide, polypeptide, or protein and a second pharmaceutically acceptable carrier, wherein the second pharmaceutically acceptable carrier has a pH of at least one pH unit above the isoelectric pH of the peptide, polypeptide or protein.

44. The method according to claim 42, wherein the non-peptidic polymeric cosmetic or therapeutic agent is selected from the group consisting of polysaccharides, polynucleotides, and synthetic polymers.

45. The method according to claim 44, wherein the polysaccharide is selected from the group consisting of hyaluronic acid, polylactic acid, heparin, heparan sulphate, dermatan sulphate and chondroitin sulphate.

46. The method according to claim 42, wherein the peptide, polypeptide or protein is selected from the group consisting of insulin, proinsulin, follicle stimulating hormone, insulin like growth factor-1, insulin like growth factor-2, platelet derived growth factor, epidermal growth factor, fibroblast growth factors, nerve growth factor, colony stimulating factors, transforming growth factors, tumor necrosis factor, calcitonin, parathyroid hormone, growth hormone, bone morphogenic protein, erythropoietin, hemopoietic growth factors, luteinizing hormone, calcitonin, glucagons, clotting factors such as factor VIIIC, factor IX, tissue factor, and von Willebrand factor, anti-clotting factors such as Protein C, atrial natriuretic factor, lung surfactant, plasminogen activator, such as urokinase or tissue-type plasminogen activator, including human tissue-type plasminogen activator (t-PA), bombesin, thrombin, enkephalinase, a collagen domain, mullerian-inhibiting agent, relaxin A-chain, relaxin B-chain, prorelaxin, Dnase, inhibin, activin, vascular endothelial growth factor, receptors for hormones or growth factors, integrin, protein A or D, a neurotrophic factor such as bone-derived neurotrophic factor (BDNF), neurotrophin-3, -4, -5, or -6 (NT-3, NT-4, NT-5, or NT-6), immunotoxins, interferon such as interferon-alpha, -beta, and -gamma; colony stimulating factors (CSFs), interleukins (ILs) such as IL-1 to IL-10; surface membrane proteins, viral antigen such as a portion of the AIDS envelope, transport proteins, homing receptors, regulatory proteins, collagen, gelatin, fibrin, keratin, elastin, laminin, bovine serum albumin (BSA), polylysine, antibodies, glycoproteins, phosphoproteins, avidin-biotin complexes and lectins.

47. The method according to claim 42, wherein the cosmetic or pharmaceutical composition further comprises an additional therapeutic or cosmetic agent.

48. The method according to claim 42, wherein the cosmetic composition comprises hyaluronic acid and BSA.

49. The method according to claim 42, wherein the cosmetic composition comprises hyaluronic acid and collagen.

50. A method for iontophoretic transdermal delivery of a polypeptide comprising at least one hundred amino acid residues, which comprises:
removing the stratum corneum from a predetermined site on the skin of a subject;
providing a system for iontophoretic transdermal delivery comprising an iontophoresis device comprising at least one agent reservoir adapted for holding a cosmetic or pharmaceutical composition, wherein the cosmetic or pharmaceutical composition comprises as an active ingredient a polypeptide comprising at least one hundred amino acid residues, and a pharmaceutically acceptable carrier, wherein the pharmaceutically acceptable carrier has a pH of at least one pH unit below or above the isoelectric pH of the polypeptide, thereby increasing the positive or negative charge of said polypeptide;
placing the agent reservoir in contact with the site on the skin of the subject; and
a) delivering said polypeptide to the subject by iontophoresis.
